(19) United States
(12) Patent Application Publication
(10) Pub. No.: US 2012/0039886 A1
(43) Pub. Date: Feb. 16, 2012

(54) USE OF AN IMMUNOGLOBULIN G (IGG) CONCENTRATE DEPLETED OF ANTI-A AND ANTI-B ANTIBODIES FOR TREATING NEONATAL JAUNDICE CAUSED BY MATERNAL-FOETAL INCOMPATIBILITY WITH RESPECT TO THE ABO SYSTEM

(75) Inventor: Mazen Elzaabi, Paris (FR)

(73) Assignee: Laboratoire Francais Du Fractionnement Et Des Biotechnologies, Les Ulis (FR)

(21) Appl. No.: 13/140,541

(22) PCT Filed: Dec. 16, 2009
(86) PCT No.: PCT/FR2009/052558

§ 371 Date: Oct. 31, 2011

(30) Foreign Application Priority Data
Dec. 17, 2008 (FR) 0807105

(51) Int. Cl. A61K 39/395; A61P 3/00

(52) U.S. Cl. 424/137.1

(57) ABSTRACT

An embodiment relates to the use of an immonuglobulin (IgG) concentrate depleted of anti-A (AcaA) and anti-B (AcaB) antibodies for producing a drug intended for treating neonatal jaundice caused by maternal-fetal incompatibility with respect to the ABO system.

### PRIORITY CLAIM

The present application is a national phase application filed pursuant to 35 USC §371 of International Patent Application Serial No. PCT/FR2009/052558, filed Dec. 16, 2009; which further claims the benefit of French patent application Ser. No. 08/07105 filed Dec. 17, 2008; all of the foregoing applications are incorporated herein by reference in their entireties.

### TECHNICAL FIELD

One or more embodiments relate to the use of an immunoglobulin G (IgG) concentrate depleted of anti-A (AcaA) and anti-B (AcaB) antibodies for manufacturing a medicament intended for the treatment of neonatal jaundice caused by maternal-foetal incompatibility with respect to the ABO system.

In the following description, the references between square brackets ([ ]) refer to the list of references presented after the examples.

### BACKGROUND

Neonatal haemolytic disease (NHD) is a disorder of the foetus or neonatus due to incompatibility between the anti-erythrocyte antibodies of the mother and the erythrocytes of the child. The anti-erythrocyte antibodies cause haemolysis already occurring in utero and, according to the gravity of the clinical picture, an occasionally severe anaemia in the child. Bilirubin being the degradation product of the haeme (constituting haemoglobin) transported in the blood, the picture may range from hyperbilirubinemia accompanying jaundice in the child, to very severe pathology with anasarca (hydrops foetalis) and delivery of a stillborn child. Untreated, hyperbilirubinemia may cause kernicterus in the neonatus.

Knowledge of the clinical picture and of the diagnostic exploration thereof is of extreme importance, all the more so since today effective means are available for preventing NHD (anti-D prophylaxis or rhesus factor) as well as effective treatments, intra-uterine/postnatal exsanguino-transfusion, UV neonatal therapy).

Nevertheless, all this is possible only if the risk is recognized in time, that is to say before pregnancy (in particular by determining the blood group of the mother and possibly of the father), at the start of pregnancy (possibly confirmation of the blood groups and search for alloantibodies, including anti-A/anti-B immune antibodies) or during pregnancy (checking the change in titer every 3 to 4 weeks). In addition, determining the blood group of the father of the child may be very useful (phenotype with indices for reaching a conclusion as to the probable rhesus genotype), since it is then possible to deduce therefrom the probable blood group of the child.

The true risk of NHD can be estimated according to the blood group of the mother or the low titer of an alloantibody and of the presumed blood group of the child, established from the blood group of the father. If the mother forms part of a blood group at risk (e.g. O or rhesus D negative), under no circumstances should checks on change in titer be abandoned.

In rare cases, when there is a constellation of similar risks, biomolecular determination of the blood group of a child from a chorial biopsy may be indicated.

ABO incompatibility occurs typically in the case of a mother in blood group O and a child in blood group A or B.

The anti-ABO antibodies of the IgM isotype, present in the natural state, do not cross the placental barrier. Although the ABO system is not yet completely developed and expressed at birth—definitive determination of the blood group should not be carried out before the age of 6 months in a child—group A or B may be detected on the erythrocytes of the foetus at an early stage of pregnancy. This is why immunization of the mother is relatively frequent even in this constellation. Moreover, anti-A and anti-B IgG immune antibodies may be induced by foreign proteins, independently of pregnancy or a prior blood transfusion. However, the anti-A or anti-B IgG antibodies almost always have a lower haemolytic capacity than similar antibodies in the case of rhesus incompatibility. Consequently the change in NHD is more discreet.

Jaundice appears in 70% of cases between 24 and 72 hours after birth. It is in all cases detected visually or instrumentally, often before the reception of the result of the cord blood, justifying the successful initiation of phototherapy. In no cases does early knowledge of the results of the group lead to a therapeutic, preventive or curative measure as long as the diagnosis of jaundice has not been posed. At the very most it gives rise to increased vigilance faced with the coloring of the neonatus.

Jaundice is by far the most frequent of symptoms observed in the neonatal period.

The first characteristic is that, unlike in the adult, jaundice in the neonatus is, in the immense majority of cases, with indirect bilirubin (BR).

This pigment, by accumulating in the organism, will concern all the organs, but in particular the liver (which, above all, takes account of this accumulation), the blood (which partly conveys and stores the pigment), the skin and the brain, with a constant potential risk of bilirubinic encephalopathy, which justifies the greatest rigor in the conduct of diagnosis and treatment.

Traditionally, children are treated by means of intravenous immunoglobulins (IVIG) and phototherapy, and in the most serious cases by exchange transfusion.

Studies have related to the treatment of new born babies afflicted by ABO and/or Rh incompatibility haemolytic disease by means of high doses of IVIG (intravenous immunoglobulins), and showed that administration of IVIG reduced the number of children needing exchange transfusions, as well as the duration of the treatment by phototherapy (Gottstein et al. (2003), Archives of Disease in Childhood; Fetal and Neonatal Edition; vol. 88, no. 1, p. F6-F10[2], which is incorporated by reference).

It has also been shown that therapy for newborn babies affected by ABO and/or Rh incompatibility haemolytic disease by means of strong doses of IVIG reduces the haemolysis, the levels of bilirubin in the serum and the need to carry out exchange transfusion (Alplay et al (1999), Acta Paediatrica, International Journal of Paediatrics; vol. 88, no. 2, p. 216-219[3], which is incorporated by reference).

However, a study has shown that the administration of IVIG to newborn babies affected by isoimmune haemolytic jaundice due to Rh and ABO incompatibilities give rise to significantly better results for the treatment of Rh isoimmunization compared with that of ABO isoimmunization. This is because the exchange transfusion requirements of newborn babies having hyperbilirubinemia due to Rh incompatibility were reduced at the end of treatment by administration of IVIG compared with treatment by phototherapy. On the other hand, in the case of newborn babies having ABO incompatibility, phototherapy and the administration of IVIG showed no difference in terms of results (Nasseri et al. (2006) Saudi Med J.; 27(12): 1827-30[4], which is incorporated by reference).

In addition, the administration of high doses of IVIG must be considered to be a risky treatment in some patients having ABO incompatibility. This is because IVIGs are concentrates of immunoglobulins issuing from human plasma, and in this regard include anti-A antibodies and anti-B antibodies.

Many scientific publications indicate that the injection of immunoglobulin G (IgG) obtained by conventional fractionation techniques, such as precipitation with ethanol, or precipitation by octanoic acid (Steinbuch et al. (1969) Rev. Franc. Et. Clin, et Biol., XIV, 1054 [5], which is incorporated by reference), may cause accidental haemolyses, including some severe ones, in patients under treatment. By way of example the publications by Buchta C. et al, Biologicals. 33, 2005, 41-48 ([6]), Wilson J. R. et al, Muscle & Nerve, 29(9), 1997, 1142-1145 ([7]), Copelan E. A. et al, Transfusion, 26, 1986, 410-412 ([8]) and Misbah S. A. et al, Drug Safety, 9, 1993, 254-262([9]), which are incorporated by reference, can be cited. A study of the effects of these IgGs on the blood of patients having haemolysis, implemented in particular by the direct Coombs test (DCT), showed that the erythrocytes are covered by immunoglobulins directed against the A, B or D antigens present on the surface thereof, thus causing haemolysis thereof.

This is why the IgGs currently available commercially are obtained from plasmas selected so as to avoid the presence of high titers of anti-A or anti-B.

According to the European Pharmacopoeia (method 2.6.20, also referred to as the indirect Coombs test (ICT), 1997, which is incorporated by reference), IVIGs must not exhibit agglutination of the A or B erythrocytes in the indirect Coombs test (ICT) in vitro at dilution 1/64 used with a solution of IgG with an initial concentration adjusted to 30 g/l. In other words, the maximum titer allowed by the European Pharmacopoeia must be less than 64 (reverse dilution (“results given as whole number—the lower part of the dilution fraction”)), in accordance with method 2.6.20 of the European Pharmacopoeia, that is to say a composition of IVIG diluted to 1/64 must not cause agglutination of the red cells (erythrocytes).

Negative results to the ICT test, that is to say an absence of agglutination of the erythrocytes, in the presence of solutions of IVIG the dilutions of which are less than the dilution of 1/64 according to the European Pharmacopoeia, demonstrate a low level of anti-A and anti-B antibodies accepted by it. However, even with concentrations of IgG giving a negative result to the test prescribed by the European Pharmacopoeia, that is to say those the dilution of which is less than 1/64, the risks of haemolytic reactions cannot be excluded ([6]).

Moreover, the American and Japanese Pharmacopoeia do not contain any provision on the need to control the residual anti-A and anti-B antibody contents.

The anti-A and anti-B antibodies are partially eliminated during the preparation of IgG concentrates by conventional methods, such as ethanol fractionation, but a residual content is observed that may be higher than the limit of the high standards of the European Pharmacopoeia. In addition, the concentrates prepared according to the method developed by the applicant and described in its patent application WO 02/092632, which is incorporated by reference, contain more of them than those obtained by ethanol fractionation. Some batches of IgG concentrates may have contents higher than the threshold accepted by the European Pharmacopoeia for each of them.

Consequently it appears necessary to have available a treatment for neonatal jaundice caused by maternal-fetal incompatibility with respect to the ABO system that can be administered to newborn babies suffering from ABO incompatibility, without fear of causing haemolysis.

### DETAILED DESCRIPTION

It was for this purpose and during its extensive research that it was surprisingly shown that a composition of immunoglobulins G having respective titers of anti-A and anti-B antibodies in accordance with a negative result to the indirect Coombs test in vitro can be used for the treatment of neonatal jaundice caused by maternal-fetal incompatibility with respect to the ABO system or neonatal haemolytic disease caused by ABO incompatibility, without causing the undesirable secondary effects found with the compositions of the prior art.

Thus an embodiment concerns a haemoglobulin G (IgG) composition for therapeutic use, comprising respective titers of anti-A and anti-B antibodies in accordance with a negative result to the indirect Coombs test (method 2.5.20 of the European Pharmacopoeia, which is incorporated by reference), as a medicament for the treatment of neonatal jaundice caused by maternal-fetal incompatibility with respect to the ABO system or neonatal haemolytic disease caused by ABO incompatibility.

“Immunoglobulins G” means, within the meaning of the present disclosure, polyclonal IgGs, which can be obtained from blood plasma or a fraction of blood plasma already enriched with IgG. The compositions or concentrates of IgG for therapeutic usage advantageously have concentrations of IgG commonly used, for example approximately between 50 g/l and 100 g/l.

“Therapeutic use” means, within the meaning of the present disclosure, use aimed at improving the state of health of the patient, for example reducing the virulence of the jaundice, or the total or partial disappearance thereof, or even curing of the patient.

“Respective titers of anti-A and anti-B antibodies” means, within the meaning of the present disclosure, the titer as defined in the European Pharmacopoeia (European Pharmacopoeia 2.6.20, which is incorporated by reference) and measured by the indirect Coombs test (ICT test), which is incorporated by reference. In other words, the titer is the dilution as from which an agglutination is detected in accordance with method 2.6. 20 of the European Pharmacopoeia. Also in other words, the titer is the dilution as from which haemolysis is observed.

The ICT test (method 2.6.20 of the European Pharmacopoeia, which is incorporated by reference) consists of a suspension of erythrocytes put in contact with the IgG composition of an embodiment, a solution of antibodies (antiglobulins) directed against the units of human IgG. These antibodies are fixed to anti-A or anti-B antibodies attached to the erythrocytes and thus enable agglutination thereof by the formation of bridges between the IgGs. The test consisting of the search for anti-A or anti-B antibodies is inspired directly by this conventional haematological serology test (indirect Coombs test).

In the context of an embodiment, method 2.6.20 of the European Pharmacopoeia can be used in the following manner: 2 identical series of dilutions of the IgG composition of an embodiment are prepared in an approximately 9 g/l solution of sodium chloride R. To each dilution of the first series, an approximately equal volume of an approximately 5% VN suspension of red cells A is added, previously washed 3 times with the sodium chloride solution. To each solution of the second series, an approximately equal volume of an approximately 5% V/V suspension of red cells B is added, previously washed 3 times with the sodium chloride solution. The suspension is left to incubate at approximately 37° C. for approximately 30 minutes and then the cells are washed with the sodium chloride solution. Each suspension is put in contact with a multipurpose human antiglobulin reagent for approximately 30 minutes. Without centrifuging the mixtures, any agglutination is sought by microscopic examination. If the IgG composition before dilution has an immunoglobulin content greater than approximately 30 g/l, a dilution to achieve this concentration of approximately 30 g/l is carried out in order to prepare the dilutions to be used in the test. Dilutions to approximately 1/64 do not present any signs of agglutination.

When the level of anti-A and anti-B antibodies is very low in the IgG concentrates, the ICT test is negative, all the more so under the conditions of the European Pharmacopoeia, given that the erythrocytes agglutination reactions no longer take place, even with the addition of human anti-IgG antibodies, since the density of anti-A and anti-b antibodies is too low to establish bridges between the erythrocytes by means of the anti-A and anti-B antibody bonds fixed to the erythrocytes and the human anti-IgG antibodies.

The European Pharmacopoeia test 2.6.20, or ICT test, is the only test recognized at a regulatory level, and all the IVIGs marketed in Europe must conform to this test, that is to say have an absence of agglutination at a dilution of approximately 1/64. The maximum titer allowed by the Pharmacopoeia must be (reverse dilution (“results given as a whole number—the lower part of the dilution fraction”)) less than 64 (<64). That is to say, at a dilution of approximately 1/64, the product tested does not cause agglutination, in accordance with what is described in method 2.6.20 of the European Pharmacopoeia, which is incorporated by reference.

“Negative result to the indirect Coombs test” means, within the meaning of the disclosure, an absence of agglutination of erythrocytes, measured in accordance with method 2.6.20 of the European Pharmacopoeia, when they are put in contact with the IgGs of the composition according to the disclosure, in the presence of multipurpose human antiglobulin.

The composition of immunoglobulins G according to an embodiment can, therefore, have, advantageously, respective anti-A and anti-B antibody titers in accordance with a negative result to the in vitro indirect Coombs test of approximately 64, which is incorporated by reference, reverse dilution (“results given as a whole number (the lower part of the dilution fraction”), that is to say negative to dilution approximately 1/64.

The immunoglobulin G composition according to an embodiment advantageously has a titer (reverse dilution (“results given as a whole number—lower part of the dilution fraction”) of approximately between 0, 0 advantageously being excluded, and 8. At these anti-A and anti-B antibody titers, the IgG composition according to an embodiment has a result in accordance with the ICT, that is to say an absence of agglutination. That is to say, at a dilution of approximately between 0 (no dilution), 0 advantageously being excluded, and 1/8, the IgG composition according to an embodiment does not cause an agglutination, in accordance with what is described in method 2.6.20 of the European Pharmacopoeia. Thus, a titer of 8 means that an agglutination is observed beyond the 8^{th} dilution of the sample (dilution to 1/8). A titer of 0 signifies no agglutination is detected, even when the sample is not diluted.

Advantageously, the anti-A antibody and anti-B antibody titers, given as a whole number (the lower part of the dilution fraction)” is less than approximately 4, or less than approximately 2. Such IgG compositions do not cause agglutination when they are diluted respectively 4 times (dilution to) or twice (dilution to).

Thus the IgG compositions (or concentrates) for implementing an embodiment have anti-A and anti-B antibody titers appreciably less than those observed in standard IgG concentrates, that is to say those obtained by ethanol fractionation and/or by the use of the purification techniques associating chromatographs without a supplementary step of eliminating the antibodies in question.

In addition the titers are appreciably below the thresholds accepted by the European Pharmacopoeia, which very significantly limits the risk of haemolysis in some receivers under treatment. Implementation of the indirect Coombs test, with IgG compositions according to the invention, may lead to negative results, even with samples of IgG as they stand, undiluted.

The IgG concentrates according to an embodiment are therefore defined by a notable absence of the anti-A and anti-B antibody active principles that are directed against the epitopes present on the erythrocytes.

It has been found that an IgG composition as described is particularly suited to the treatment of neonatal jaundice caused by maternal-foetal incompatibility with respect to the ABO system.

Advantageously, the immunoglobulin G composition according to an embodiment can therefore have, advantageously, respective anti-A and anti-B antibody titers equal to approximately 64, reverse dilution (“results given as a whole number (the lower part of the dilution fraction”), or approximately between 0 and 8, or less than approximately 4, for example less than approximately 2. Advantageously, in these cases, the Coombs test is implemented with an IgG solution with an initial concentration adjusted to approximately 30 g/l.

“Neonatal jaundice caused by maternal-foetal incompatibility with respect to the ABO system” means, within the meaning of the present disclosure, jaundice accompanying neonatal haemolytic disease (NHD) caused by incompatibility with respect to the ABO system (also referred to as ABO haemolytic disease, neonatal haemolytic disease with ABO incompatibility or isoimmune haemolytic jaundice due to ABO incompatibility). This jaundice is due to an accumulation of bilirubin in several organs, the bilirubin being the product of degradation of the haeme transported in the blood.

More particularly, the patients to which the IgG composition described may be administered may be premature newborn babies or newborn babies born at term, for example from birth up to the 28^{th} day after birth. These babies generally exhibit hyperbilirubinemia due to ABO haemolytic disease, are positive to an antiglobulin neonatal test (indirect Coombs test) and have a high reticulocyte count (greater than or equal to 10%). These infants may be boys or girls.

The composition of an embodiment may therefore also be use as a medicament for treating neonatal haemolytic disease caused by ABO incompatibility.

The IgG compositions, IgG concentrates, as defined above may have a very low polyreactive IgG content, which may for example be approximately between 0.01% and 0.1%, in particular approximately between 0.7% and 0.1%. In this context, the polyreactive IgG content signifies a molar percentage or percentage by weight. This content may be determined by the methods described in patent application EP 1 059 088, which is incorporated by reference.

For example, the immunoglobulin G composition according to an embodiment may advantageously have respective anti-A and anti-b antibody titers of approximately 64, reverse dilution (“results given as a whole number (the lower part of the dilution fraction”), or approximately between 0 and 8, or less than approximately 4, for example less than approximately 2, the Coombs test being implemented with an IgG solution with an initial concentration adjusted to approximately 30 g/l, and a polyreactive IgG content that may for example be approximately between 0.01% and 0.1%, in particular approximately between 0.7% and 0.1%.

“Polyreactive IgGs” means, within the meaning of the present disclosure, an IgG fraction contained in the IgG composition defined above, corresponding to the sum of the natural antibodies that do not result from deliberate immunization and express variable affinities for self antigens, anti-idiotypical antibodies (that is to say directed against the variable region of other antibodies), and antibodies that have become polyreactive following treatments received during different steps of the purification method of the IgG composition defined above. Advantageously, the IgG composition used in an embodiment is distinguished from the other IgG compositions available commercially through the almost total absence of polyreactivity. However, it has been discovered entirely surprisingly that this almost total absence of polyreactivity in the IgG compositions, combined with very low anti-A and anti-B antibody levels in these compositions, is an important feature for the treatment of neonatal jaundice caused by maternal-fetal incompatibility with respect to the ABO system or neonatal haemolytic disease caused by ABO incompatibility. Thus the IgG composition used in an embodiment is effective as a medicament for treating neonatal jaundice caused by maternal-foetal incompatibility with respect to the ABO system or neonatal haemolytic disease caused by ABO incompatibility, while avoiding undesirable reactions with regard to erythrocytes (in particular by anti-A and anti-B antibody impoverishment) and reducing the undesirable secondary reactions that result from the presence of polyreactive IgGs (in particular fever, nausea and cephalea).

The composition of an embodiment can also include one or more stabilizers.

“Stabilizer” means, within the meaning of the present disclosure, a compound for preserving the IgG composition over time. The stabilizer can in particular enable the IgG composition to be kept for a specified period. Moreover, the stabilizer is advantageously compatible with therapeutic use.

The stabilizer may advantageously be one of those described in patent application WO 2204/091656, which is incorporated by reference, namely a mixture of alcohol sugar, such as mannitol, sorbitol, or isomers thereof, glycine and a non-ionic detergent, such as Tween™80, Tween™20, Triton □X100 or Pluronic □F68, all three being acceptable compounds on a pharmaceutical level.

The final concentrations of mannitol in the IgG composition may be approximately between 30 g/l and 50 g/l, and those of glycine approximately between 7 g/l and 10 g/l. The concentrations of these compounds represent the final concentrations in the IgG compositions.

Advantageously, the concentrations of the formulation have been determined in order to stabilize the liquid and/or lyophilised forms. The composition of an embodiment can be administered intravenously, or subcutaneously. For this purpose, the IgG concentrates according to an embodiment are virally protected, for example by a conventional solvent/detergent treatment known from the prior art, using for example a Tween 80/TnBP or Triton X 100/TnBP, mixture, and/or undergo filtration steps in order if necessary to eliminate the viruses and/or other macromolecules that have not been eliminated by the solvent/detergent viricidal treatment, such as prion, the agent responsible for transmissible spongiform encephalopathies.

The IgG concentrates according to an embodiment can also be subjected to a nanofiltration step.

The composition of an embodiment can be formulated so as to be in liquid or lyophilised form in the presence of suitable stabilizers, or be stored while awaiting subsequent use.

Advantageously, the composition can be injected intravenously.

In an embodiment, the composition may be a solution for injection, for example a solution of normal human immunoglobulin for injection for intravenous use at approximately 5 g/100 ml (5%).

Advantageously, the solution for injection can be dosed at approximately 1 g/100 ml (1%), or at approximately 2 g/100 ml (2%), or at approximately 3 g/100 ml (3%) or at approximately 4 g/100 ml (4%), or at approximately 5 g/100 ml (5%).

Advantageously, the IgG doses of the injectable solution can be approximately between 1 g/100 ml and 5 g/100 ml, or approximately between 2 g/100 ml and 5 g/100 ml, approximately between 3 g/100 ml and 5 g/100 ml, or equal to approximately 5 g/100 ml.

The composition for implementing an embodiment can be administered concomitantly with or consecutively to treatment by phototherapy, for example at a quantity of approximately between 500 mg/kg and 2000 mg/kg. It may be possible to commence with an administration of approximately 500 mg/kg and then, if the level of bilirubin does not decrease (which can be tested by a known bilirubinemia test), to increase the dose in steps of approximately 500 mg/kg until the level of bilirubin is normalized. The administration can be repeated.

The composition for implementing an embodiment can be administered without parallel phototherapy. The quantity administered can be approximately between 500 mg/kg and 2000 mg/kg. It may be possible to commence with an administration of approximately 500 mg/kg and then, if the level of bilirubin does not decrease (which can be tested by a known bilirubinemia test), to increase the dose in steps of approximately 500 mg/kg until the level of bilirubin is normalized. This administration can be repeated.

A composition suited to the implementation of an embodiment can be identical to that described in the document WO 2007/077365, which is incorporated by reference.

The IgG composition can be obtained by any conventional method.

In particular, the composition can be obtained by an embodiment of a method including the following steps:

a) preparing an IgG composition by ethanol fractionation and/or by chromatographic separation, comprising a viral inactivation step,

b) immunoaffinity chromatography by percolation of the IgG composition on a mixture of carriers, the matrices of which are grafted with oligosaccharide groups antigenically similar to the A and B blood groups, and

c) elimination filtration of viruses and/or particles with a size greater than approximately 20 nm.

Advantageously, such a method is described in the document WO 2007/077365, which is incorporated by reference.

Such a method can very advantageously be implemented on an industrial scale. In addition, the combination of steps resulting in the preparation of IgG compositions and a specific step of elimination of anti-A and anti-B antibodies may make it possible to obtain an IgG composition, for therapeutic use, also, for example, comprising a level of polyreactive IgGs less than approximately 0.1% with respect to the total IgG content. In addition, such a composition includes a titer of undesirable AcaAs and AcaBs much less than the lower limit value of the test described in the European Pharmacopoeia, that is say below approximately 64 (reverse dilution (“results given as a whole number—the lower part of the dilution fraction”)) and even giving a negative result by implementing the ICT test with such undiluted samples, that is to say a titer equal to approximately 0.

Step a) of the method can in itself be a method of obtaining IgG concentrates such as those well known to persons skilled in the art. It is a case of an ethanol fractionation developed by Cohn et al. (Cohn et al. 1946, J. Am. Chem. Soc. 68, 459; Oncley et al. 1949, J. Am. Chem. Soc. 71, 541 [11], which is incorporated by reference) or a chromatographic separation as described, for example, in EP 0 703 922 and WO 99/64462, which are incorporated by reference. The methods described in the patent applications WO 94/29334 and WO 02/092632, which are incorporated by reference, may be especially preferred, and in particular the one described in WO 02/092632. In this case, step a) of the method of an embodiment includes a prepurification by precipitation of lipid contaminants from a blood plasma or a fraction of blood plasma enriched with IgG, a single chromatography on an anion exchange resin carrier carried out at alkaline pH, and a selective elution of the IgGs in one step by a suitable buffer at a pH of approximately between 4 and 7.

“Lipid contaminants” means, within the meaning of an embodiment, the constituents of the plasma other than immunoglobulins.

“Fraction of blood plasma enriched with IgG” means, within the meaning of the present disclosure a plasma fraction that has already undergone purification steps, so as to increase the IgG concentration of this fraction.

“Single chromatography” means, within the meaning of the present disclosure, a chromatography step that is not repeated subsequently.

“Selective elution of the IgGs in one step” means, within the meaning of the present disclosure, an elution step for eluting the major part of the immunoglobulins G.

For the purpose of an embodiment, the buffers for selectively eluting the IgGs in one step can be any conventional buffer.

Step a) of an embodiment includes a viral inactivation treatment, for example performed by a solvent/detergent, as described by Horowitz in U.S. Pat. No. 4,764,369, which is incorporated by reference. It will in particular be carefully implemented, where necessary before the subsequent chromatographic step for eliminating in particular the chemical residues of this treatment.

This concentrate is then subjected to an immunoaffinity chromatographic step on a mixture of two grafted carriers of groups antigenically similar to the blood groups A and B, for example on a column filled with a mixture of carriers. The chromatographic carrier may consist of a matrix made from crosslinked natural polymer of the agarose type, on which spacers or coupling arms are grafted, being in their turn grafted with oligosaccharides advantageously representing trisaccharides (oligosaccharides) corresponding to the epitopes of blood groups A and B.

“Oligosaccharide groups antigenically similar to blood groups A and B” means, within the meaning of the present disclosure, oligosaccharide groups recognized by the same antibodies or the same immunoglobulins as blood groups A and B.

In particular, very good results are obtained using such a carrier, the trisaccharides of which, corresponding to the epitope of blood group A, have the structure N-acetylgalactosamine (GalNAc)—Galactose (Gal)—Fucose (Fuc), and those corresponding to the epitope of blood group B, have the structure Galactose-Galactose-Fucose. Such a carrier very advantageously represents a gel or resin commercially available under the name GLYCOSORB ABO coming from Glycorex Transplantation AS (Sweden).

By way of example, of this carrier is used, the trisaccharide corresponding to the epitope of blood group A has the following structure:

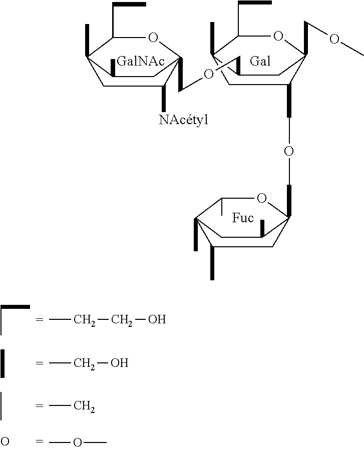

N-acetylgalactosamine (GalNAc)

Galactose (Gal)

Fucose (Fuc)

By way of example, if this carrier is used, the trisaccharide corresponding to the epitope of blood group B has the following structure:

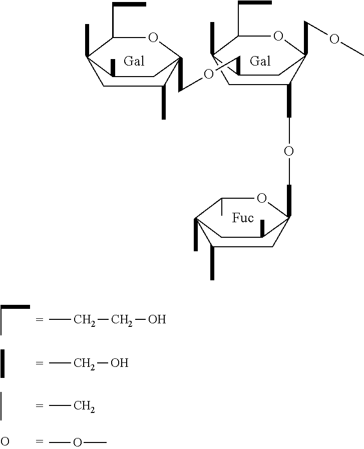

“Carrier” means, within the meaning of the present disclosure, an inert material serving to support the matrix.

Advantageously, a matrix carrying a type of functional group, namely a type of oligosaccharide groups, is grafted to a carrier.

These carriers are well known.

The matrix may be any suitable matrix. These matrices are well known. Sepharose matrices, for example Glycosorb ABO (Glycorex Transplantation), can in particular be given as an example.

It is possible to use the Glycosorb ABO matrix (Glycorex Transplantation), which is grafted with trisaccharides of blood groups A and B.

“Mixture of carriers” means, within the meaning of the present disclosure, the mixture of carriers some of which carry the matrix grafted with the oligosaccharides antigenically similar to blood group A, and others of which carry the matrix grafted with the oligosaccharides antigenically similar to blood group B.

The different types of matrix can therefore be found in variable proportions.

Advantageously, the mixture of carriers grafted with groups antigenically similar to blood group A and blood group B is in a respective proportion of approximately between 25/75 and 75/25 (v/v). It may be in fact possible to adjust the proportion of the two carriers in the column to the population of donors according to the distribution of the blood groups thereof. In the context of habitual use, the column will, for example, be filled with a mixture of approximately 50/50 (v/v) of each specific carrier above. It may be possible to use analytical columns approximately 15 to 25 cm long and approximately 0.5 to 1 cm in diameter. In the case of implementation on a pilot scale, it may be possible to use columns approximately 40 to 60 cm long and approximately 40 to 60 mm in diameter. In this case, it may be possible to load the column with approximately 600 ml of immunoaffinity carrier.

Such a carrier can be stored in approximately 1 M NaOH between two use cycles. Before use it is washed with water.

The immunoaffinity choromatographic column can then be loaded with IgG concentrate, for example, to the extent of approximately 0.2 to 4 litres, in particular approximately 1 to 2 litres, per millilitre of carrier. The specificity of such a carrier does not require prior conditioning of the IgG fraction, that is to say any fraction of concentrate of IgG obtained by the plasma fractionation techniques of the prior art may suit.

Percolation of the concentrate does not involve the elution mechanism. Consequently, whatever the way in which the IgG concentrate is obtained, it can be percolated through the column, optionally by means of a pump. This percolation enables the AcaAs and AcaBs and the polyreactive IgGs to be retained. Advantageously, the column is then washed with water in order to recover the IgGs still present in the dead volume of the column.

After percolation of the IgG concentrate, a fraction of IgG depleted in AcaA and AcaB, as well as in polyreactive IgGs issuing from the manufacturing process, is obtained. This is because the AcaAs and AcaBs are retained on their antigen unit of the chromatographic carrier, which modifies the conformation thereof.

The affinity of these polyreactive IgGs retained in a secondary fashion is much greater than that of the AcaAs and AcaBs. It is possible to elute them in a fractionated manner, after passage of the IgGs, by use of an elution buffer containing for example an alkaline-earth metal salt with a concentration of approximately between 0.1 and 1.5 M, at a pH of approximately 3-8.6.

The chromatographic column and the carrier can then be washed with an acid solution, such as glycine-HCl, approximately pH 2.8, for desorption of the AcaAs and AcaBs retained on the carrier. This carrier is then rinsed with water and treated with an approximately 1 M NaOH solution.

The IgG concentrate highly depleted of AcaA and AcaB is then subjected to a filtration for elimination of viruses resistant to the solvent/detergent treatment and/or other particles with a size greater than approximately 20 nm, such as prions, the IgG polymers generated during steps of its manufacture, the lipopolysaccharides in micelles, the nucleic acids and the aggregated proteins. Such treatment advantageously represents nanofiltration, implemented by filters of porosity decreasing from approximately 100 to 15 nm, in particular on three filters disposed in series and decreasing retention thresholds, of approximately 100, 50 and 20 nm.

Advantageously, the method comprises a viral inactivation step.

“Viral inactivation” means, within the meaning of the present disclosure, any method or step for inactivating, that is to say effectively denaturing, the viral particles while respecting the functionality of the plasma proteins.

The viral inactivation methods are well known. Among these methods, a viral inactivation step can the chosen from: the solvent/detergent step or pasteurization.

Advantageously, the viral inactivation step can be performed by means of a solvent/detergent step.

The IgG fraction thus harvested is already sufficiently concentrated, and can then undergo additional concentration steps by ultrafiltration and sterilizing filtration.

The method can comprise, after step b), steps of concentration by ultrafiltration and sterilising filtration.

Advantageously, the sterilising filtration step can be performed by nanofiltration.

The method can comprise, after step c), an additional step of adding stabilizers in order firstly to ensure stability of the IgG concentrates during preservation over time and secondly to allow lyophilisation preventing the denaturation of the IgGs in the various phases associated therewith. For example, a pharmaceutically acceptable single stabilizing formulation will be added, meeting the objective of ensuring stabilizing of the two envisaged preservation forms of the IgGs at the same time, namely in liquid or lyophilised form, and to preserve or even improve the therapeutic efficacy of these IgGs, as described in patent application WO 2004/091656, which is incorporated by reference.

The IgG compositions are optionally subjected to a subsequent step of concentration by ultrafiltration, and then to a sterilizing filtration, and can be packaged in flasks and kept at temperatures of around 4° C.

An analysis method can be used for analysing the anti-A and anti-B antibodies of the IgG composition described according to an embodiment.

Such a method of analyzing the anti-A and/or anti-B antibodies in the IgG concentrates can comprise the steps of:

a) preparing and calibrating a suspension of erythrocytes of blood group 0 rhesus+,

b) preparing monoclonal anti-D antibody solutions in a range of concentrations from approximately 0 to 200 ng/ml in a biologically acceptable buffer,

c) putting said erythrocytes in contact with the monoclonal anti-D antibody solutions and incubating the erythrocytes mixtures thus obtained for a predetermined period,

d) adding to each mixture of erythrocytes a fragment of human anti-IgG antibodies F(ab′)2 marked by means of a fluorochrome and incubating said erythrocytes,

e) subjecting each mixture of erythrocytes obtained at step d) to flow cytometry,

f) producing a standard curve of the anti-D monoclonal antibody concentration as a function of the fluorescence.

It may be then possible to analyze the anti-A and anti-B antibodies by using the following method:

a) preparing and calibrating a suspension of erythrocytes of blood group A, B−,

b) preparing solutions of monoclonal anti-D antibodies in a range of concentrations from approximately 0 to 200 ng/ml in a biological acceptable buffer,

c) putting said erythrocytes in contact with samples of solutions of IgG, and incubating the mixtures of erythrocytes thus obtained for a predetermined period,

d) adding to each mixture of erythrocytes a fragment of F(ab′)2 human anti-IgG antibodies marked by means of a fluorochrome and incubating said erythrocytes,

e) subjecting each mixture of erythrocytes obtained at step d) to flow cytometry,

g) determining the anti-A and/or anti-B antibody titer in the IgG concentrates by means of the standard curve established for apportioning the anti-D, advantageously expressed in nanograms/millilitre.

One way of implementing such a method of determining the anti-A and/or anti-B antibody titer may comprise the preparation of an approximately 1% (v/v) erythrocytes suspension of blood group A, B and/or 0 in a PBS buffer, with a pH of approximately between 7.0 and 7.4, containing approximately 0.8 to 1.5% by weight bovine serum albumin BSA. The erythrocytes of the suspension are counted in a normal flow cytometry device, use of which is known, and then so as to calibrate the suspension at approximately 37 to 43.106 erythrocytes/ml of suspension. Monoclonal anti-D antibody solutions are prepared, the concentrations of which are included in the range from approximately 0 to 200 ng/ml of buffer, for example a PBS buffer, with a pH of approximately between 7.0 and 7.4, containing where applicable approximately 0.8 to 1.5% by weight bovine serum albumin BSA. Each solution thus prepared is analysed by absorptiometry in order to determine the molar extinction coefficient thereof ( ).

The IgG compositions are then adjusted to a concentration in the range of values from approximately 1 to 5 mg/ml, for example approximately 1 mg/ml, by means of a PBS buffer, with a pH of approximately between 7.0 and 7.4, containing approximately 0.8% to 1.5% by weight bovine serum albumin BSA.

A volume of approximately 50 to 100 μl of the suspension of erythrocytes of each blood group is placed in each well of a microplate, for example with approximately 96 wells, and then approximately 50 to 100 μl of solutions of IgG in this suspension of erythrocytes, or approximately 50 to 100 μl of anti-D antibody solutions in this suspension of erythrocytes. The whole is put to incubate for periods of approximately between 1 hour 30 minutes and 2 hours 30 minutes, for example approximately 2 hours, at temperatures normally approximately between 30° C. and 40° C., for example 37° C.

The different mixtures of erythrocytes thus obtained may then be washed with the PBS buffer containing the previous BSA and is centrifuged, and then there is added, to each mixture of erythrocytes, contained in a microplate well, approximately 50 to 100 μl of human anti-IgG goat F(ab′)2 antibodies marked with a fluorochrome, such as for example phycoerythrine, present in the PBS buffer and previously defined BSA.

Incubation of the whole is implemented for approximately 20-30 minutes away from light.

The different mixtures of erythrocytes thus obtained are then washed and subjected to flow cytometry implemented with any suitable commercially available apparatus comprising a device for fluorescence detection of the compounds analyzed.

The mean fluorescence intensity (MFI) is reported according to the anti-D monochlonal antibody concentration and the straight-line regression equation is obtained by means of Excel® software. Then, for each sample, the equivalent anti-D antibody concentration is obtained using the linear straight-line regression equation. The samples having been analyzed in triplicate, the concentration mean is established and the coefficient of variation is calculated by Excel® software.

The anti-A and anti-B antibody content in the IgG concentrates according to an embodiment is deduced therefrom, which is the one advantageously given previously.

A method of analyzing the AcaAs and AcaBs of the above IgG concentrates is implemented by flow cytometry adapted to the context of an embodiment, the principle of which is based on the use of human erythrocytes of group A or B, according to the specific determination of the titer of the AcaAs and AcaBs required, using the detection of a fluorescence signal proportional to the content of these antibodies.

Such an analysis method includes the steps of:

a) preparing and calibrating a suspension of erythrocytes of blood group A or B,

b) putting said erythrocytes in contact with diluted samples of IgG solutions, and incubating the mixture thus obtained for a predetermined period,

c) incubating said erythrocytes in the presence of an anti-IgG antibody marked by means of a fluorochrome, and

d) subjecting the suspension of erythrocytes obtained at step c) to flow cytometry.

An approximately 1% (v/v) suspension of erythrocytes of blood group A or B is prepared in a PBS buffer, with pH of approximately 7.0 and 7.4, containing approximately 0.8% to 1.5% by weight bovine serum albumin (BSA). The erythrocytes of the suspension are counted in a normal flow cytometry device, use which is known, then so as to calibrate the suspension at approximately 37 to 43.10 erythrocytes/ml of suspension.

A volume of approximately 50 to 100 μl of the suspension is placed in each well of an approximately 96-well microplate, and then approximately 50 to 100 μl of different solutions of IgG diluted two by two from a solution of approximately 30 gl until an approximately 0.234 g/l solution of IgG is obtained.

The whole is put to incubate for periods of approximately between 1 hour 30 minutes and 2 hours 30 minutes, for example 2 hours, at temperatures normally approximately between 30° C. and 40° C., for example 37° C.

The erythrocytes are then washed with the PBS buffer containing the previous BSA and is centrifuged, and then approximately 50 to 100 μl of human anti-IgG goat F(ab′)2 antibody marked with a fluorochrome, such as for example phycoerythrin, is added to each well.

Incubation of the whole (step c)) is implemented for approximately 20-30 minutes away from light.

The suspension thus obtained is then washed and subjected to flow cytometry implemented with any suitable commercially available apparatus including a device for fluorescent detection of the compounds analysed.

By way of example, the anti-A and B antibody contents of three IgG concentrates named B1, B2 and B3, prepared respectively by ethanol fractionation according to the Cohn method (B1), in accordance with patent application WO 02/092632 (B2) and according to patent application WO 02/092632, which are incorporated by reference, followed by immunoaffinity chromatography (B3) for anti-A and anti-B antibody depletion, are presented in the following table 1. The results are presented with respect to the reference anti-A and anti-B antibody titer of sample B1, the proportion of these antibodies having been arbitrarily fixed at 1, by way of reference.

TABLE 1

| Samples | Anti-A antibody content | Anti-B antibody content |
|---|---|---|
| B1 | 1 | 1 |
| B2 | 3.65 | 3.85 |
| B3 | 0.68 | 0.52 |

The results of this table show first of all that the anti-A and anti-B antibody contents of the IgG concentrates (B1), prepared according to the Chon method, contain approximately four times less of them than the IgG concentrates (B2) prepared according to the method described in WO 02/092632, which is incorporated by reference. In addition, the subsequent treatment of these IgG concentrates by specific immunoaffinity columns reduces the anti-A antibody titer by a factor of around 5 and a factor of around 7 with regard to anti-B antibodies (b3).

Another method of determining the anti-A and anti-B antibody contents that can advantageously be used consists of lysis by the in vitro complement, which is known, but which has been specifically developed for the requirements of an embodiment. Such an embodiment of analysis method includes the steps of:

a) proceeding with a radiomarking of a suspension of papainated erythrocytes chosen from blood groups A, B, AB and O, previously counted, by a suitable radioactive marker,

b) putting the radiomarked erythrocytes in contact with samples of IgG concentrates in a predetermined volume,

c) adding a volume identical to that of step b) of normal serum of blood group AB,

d) incubating the mixture obtained at step c) for a predetermined period, and

e) measuring the radioactivity of the incubated solution thus obtained.

An approximately 1% (v/v) suspension of papainated erythrocytes of blood group A, B, AB or O is prepared, and is then counted in a Malassez cell in order to obtain approximately 106 erythrocytes. approximately 100 μCi of 51 Cr is added (1 volume for 1 volume of erythrocytes). The whole is incubated for approximately between 1 and 2 hours, and the radiomarked erythrocytes are then washed approximately between 4 and 6 times.

The radiomarked erythrocytes are then put in contact with samples of IgG concentrates, at a concentration for example of approximately between 1 and 3 mg/ml, for example approximately 1.2 mg/ml, for approximately 4-6.106 radiomarked erythrocytes, in a volume for example of approximately 100 μl.

An approximately identical volume to the previous one, for example approximately 100 μl, of normal serum of blood group AB is then added to the previous mixture in order to add the different factors of the complementary method.

The reaction mixture thus obtained is then incubated, preferably for periods of approximately between 3 and 5 hours, for example approximately 4 hours, at temperatures normally approximately between 30° C. and 40° C., for example approximately 37° C.

The reaction mixture is may then be centrifuged, and a measurement is carried of the radioactivity of the incubated solution using suitable commercially available devices. The measured radioactivity of the solution is proportional to the degree of haemolysis of the erythrocytes treated and consequently to the anti-A and anti-B antibody content.

By way of example, the degree of haemolysis of the erythrocytes of blood groups A, B and AB obtained considering an IgG concentrate of an embodiment (B3) and an IgG concentrate of the prior art (C1) having the lowest haemolysis levels among the commercially available concentrates, are indicated in the following Table 2.

TABLE 2

| Degree of haemolysis | | |
|---|---|---|
| of erythrocytes (5) | B3 | C1 (prior art) |
| Group A | 6 | 13 |
| Group B | 5 | 11 |
| Group AB | 6 | 13 |

Another embodiment is the use of a composition as defined above for manufacturing a medicament intended for treating neonatal jaundice caused by maternal-foetal incompatibility with respect to the ABO system.

All the aspects of an embodiment mentioned above are adapted to this use and form part of this other embodiment.

Another embodiment is a method of treating neonatal jaundice caused by maternal-fetal incompatibility with respect to the ABO system or ABO neonatal haemolytic disease, including the administration of an IgG composition as previously defined.

In this embodiment, the composition may be a solution for injection, for example a solution of normal human immunoglobulin for injection for intravenous use at approximately 5 g/100 ml (5%).

Advantageously, the solution for injection can be dosed at approximately 1 g/100 ml (1%), or approximately 2 g/100 ml (2%), or approximately 3 g/100 ml (3%), or approximately 4 g/100 ml (4%), and advantageously up to approximately 5 g/100 ml (5%).

Advantageously, the IgG doses of the injectable solution can be approximately between 1 g/100 ml and 5 g/100 ml, or approximately between 2 g/100 ml and 5 g/100 ml, or approximately between 3 g/100 ml and 5 g/100 ml.

The composition for implementing an embodiment can be administered concomitantly with or consecutively to phototherapy treatment, for example at a quantity of approximately between 500 mg/kg and 2000 mg/kg. This administration may be repeated.

All the aspects of an embodiment described above are adapted to this treatment method and form part of this other embodiment.

Other advantages can also appear from a reading of the following examples.

### EXAMPLES

### Example 1

### Preparation of IgG Composition Depleted in Anti-A and Anti-B Antibodies

A sample of approximately 40 g/l IgG concentrate (B2) is obtained by implementing the method described in WO 02/092632, which is incorporated by reference.

A chromatographic column approximately 50 cm long and approximately 44 mm in diameter is filled with an approximately 50/50 (v/v) mixture of Glycosorb ABO carrier grafted with trisaccharides corresponding to the epitopes of blood group A and blood group B, and is then subjected to a prior washing step by approximately 1200 ml of water.

IgG concentrate B2 is injected at the rate of approximately 0.2 l/ml of carrier by means of a pump. Once this volume has been percolated through the column, the latter is washed by a minimum volume of water for injectable preparation (IPP) in order recover the IgGs present in the dead volume of the column.

An IgG concentrate B3 at approximately 40 g/l depleted in AcaA, AcaB and polyreactive IgGs is recovered, and is subjected to ultrafiltration so that the concentration is at approximately 60 g/l and to a virus-elimination nanofiltration on three filters arranged in series and with decreasing retention thresholds of approximately 100, 50 and 20 nm.

The dissolution of the stabilizing excipients consisting of a mixture of glycine at approximately 7 g/l, mannitol at approximately 30 g/l and approximately 20 ppm of Tween 80 in the IgG concentrate at approximately 60 g/l is followed by adjustment of the IgG concentration to approximately 50 g/l by means of water IPP, and then the concentrate is filtered sterilely and distributed in flasks.

### Example 2

### Quantification of the Anti-A/Bs in the IVIGs

1) Principle of the Dosing

1.1) Preparation of the Human Erythrocytes

The suspensions of human erythrocytes of group A rhesus+, B rhesus+ or O rhesus+ are normalized to the concentration of approximately 40×106 erythrocytes/ml in PBS buffer+approximately 1% BSA at pH ˜7.4.

1.2) Preparation of the Monoclonal Anti-D Range

A preparation of monoclonal anti-D (called R297) is dosed for optical density (OD) at approximately 280 nm against its PBS buffer, pH approximately 7.4. The molar extinction coefficient ( ) of the protein is calculated with respect to its amino acid composition and the concentration (C) of monoclonal anti-D is obtained by applying the formula C=OD/with approximately 1=width of the vessel for measuring the OD.

A range of approximately 0 to 200 ng/ml of anti-D monoclonal antibody is produced at approximately 12 points (200; 150; 100; 75; 50; 25; 12.5; 6.25; 3.13; 1.56; 0.78 and 0 ng/ml).

1.3) Preparation of Immunoglobulin Solutions

Different intravenous immunoglobulins, available commercially, were studied. The main characteristics of these immunoglobulins are detailed in the following table:

| Designation | Supplier | Concentration |
|---|---|---|
| Polyvalent | A | 50 g/l |
| immunoglobulin | | |
| Polyvalent | B | 50 g/l |
| immunoglobulin | | |
| 10% intravenous | C | 100 g/l |
| human immunoglobulin | | |
| IgNG 2(*) | LFB | 50 g/l |
| IgNG 1(**) | LFB | 50 g/l |
| Tegeline □ | LFB | 50 g/l |

(*)produced by implementation of the method described in WO 02/092632, which is incorporated by reference, followed by the immune affinity step described in example 1.
(**)produced by implementing the method described in WO 02/092632, without an immune affinity step as described in example 1.

The different preparations of immunoglobulins are adjusted to the concentration of approximately 1 mg/ml by means of PBS buffer+ approximately 1% BSA at pH ˜7.4.

1.4) Sensitization of the Erythrocytes

In a round-bottom microplate, the following are deposited in wells:
approximately 50 μl of the suspension of erythrocytes A rhesus+, B rhesus+ or O rhesus+ at approximately 40×106 erythrocytes/ml,
approximately 50 μl of the anti-D range or approximately 50 μl of the samples (IgIV) to be analyzed. The samples to be analyzed are deposited in triplicate.

The plates are then incubated for approximately 2 hours at approximately 37° C. under agitation.

1.5) Washings

The plates are centrifuged for approximately 1 minute a approximately 770 g. The supernatant is separated by turning over and then approximately 200 μl of PBS plus approximately 1% BSA is added in each well. The operation is repeated 3 times.

1.6) Adding of Conjugate and Washings

An F(ab′)2 of human anti-IgG goat (Fc specific) marked with phycoerythrin (PE) (Beckman Coulter ref: PN IM0550, which is incorporated by reference) is diluted to approximately 1/20 in the PBS buffer+approximately 1% BSA pH ˜7.4 and then approximately 50 μl of the solution is deposited in each well. The plate is then incubated for approximately 20 to 30 minutes at ambient temperature away from light. 3 successive washing are carried out as described in paragraph 1-5).

1.7) Reading with Flow Cytometry

The erythrocytes suspensions are read by flow cytometry (Beckman Coulter FC500, which is incorporated by reference) according to a suitable program.

The reading is carried out over approximately 50,000 events and the apparatus automatically calculates the mean fluorescence intensity (MFI) of each range point or sample.

1.8) Interpretation of the Results

The MFI is reported according to the anti-D monoclonal antibody concentration and the straight-line regression equation is obtained using Excel® software. Then, for each sample, the anti-D antibody equivalent concentration is obtained using the linear straight-line regression equation. The samples having been analyzed in triplicate, the mean of the concentration is established and the coefficient of variation is calculated by Excel software.

2) Results:

2.1) Anti-A Antibody Concentration

| | | Erythrocytes A | |
|---|---|---|---|
| | Erythrocytes O | rhesus + (ng Ig anti- | |
| Name | rhesus + | A/mg of Ig) | CV % |
| Polyvalent | ns | 55.4 | 4.5 |
| immunoglobulin, A | | | |
| Polyvalent | ns | 44.4 | 3.4 |
| immunoglobulin, B | | | |
| 10% intravenous | ns | 117.9 | 14.6 |
| human | | | |
| immunoglobulin, C | | | |
| IgNG 2 | ns | 22.2 | 5.5 |
| IgNG 1 | ns | 119.8 | 4.9 |
| TEGELINE | ns | 35.6 | 5.0 |

ns = not significant

2.2) Concentration of Anti-B Antibodies

| | | Erythrocytes B | |
|---|---|---|---|
| | Erythrocytes O | rhesus + (ng Ig anti- | |
| Name | rhesus + | B/mg of Ig) | CV % |
| Polyvalent | ns | 64.0 | 0.9 |
| immunoglobulin, A | | | |
| Polyvalent | ns | 42.4 | 5.1 |
| immunoglobulin, B | | | |
| 10% intravenous | ns | 89.0 | 20.5 |
| human | | | |
| immunoglobulin, C | | | |
| IgNG 2 | ns | 16 | 9.9 |
| IgNG 1 | ns | 155.2 | 4.8 |
| TEGELINE | ns | 44.2 | 8.0 |

ns = non significant

3) Conclusions:

The affinity step actually contributes to the elimination of the anti-A and anti-B antibodies. Among different immunoglobulins studied present on the market, the product IgNG2 is the product containing the fewest anti-A antibodies and anti-B antibodies.

### Example 3

### Quantification of the Anti-As and Anti-Bs of the IgG B Concentrate Obtained at Example 1

An approximately 1% (v/v) suspension of erythrocytes of blood group A is prepared in a PBS buffer, pH approximately 7.4, containing approximately 1% by weight bovine serum albumin BSA. approximately 50 μl of the suspension of erythrocytes is taken off and introduced into a tube for a Beckmann Coulter Epics XL flow cytometer along with approximately 50 μl of an internal marker solution measuring the flow. The suspension is calibrated at approximately 40.10⁶ erythrocytes/ml.

Eight batches of IgG solutions are prepared by successive dilution by a factor of approximately 2 of the IgG concentrate (v/v) (B3) obtained at example 1, the most concentrated batch being at approximately 30 g/l, the most diluted at approximately 0.234 g/l. Next a volume of approximately 50 μl of the suspension is placed in each well of an approximately 96-well microplate, and then approximately 50 μl of the different IgG solutions diluted. The whole is put to incubate for approximately 2 hours at a temperature of approximately 37° C., under agitation.

Each well is then washed with approximately 200 μl of PBS buffer containing some of the previous BSA and the microplate is centrifuged for approximately 1 minute at approximately 2000 revolutions/min. After elimination of the supernatant, approximately 50 μl of a solution diluted to approximately 1/20 of PBS-BSA of human anti-IgG goat F(ab′)2 antibody marked with phycoerythrin fluorochrome (Beckman-Coulter) is added in each well.

Incubation of the whole is implemented for approximately 30 minutes away from light.

The suspension thus obtained is then washed as previously.

The remainder of each well is taken up by approximately 100 μl of PBS-BSA. The volume contained in each well of the microplate is transferred into a tube in which next approximately 500 μl of liquid from an isoflow duct (Coulter) is then added and is subjected to flow cytometry implemented with the Beckman Coulter Epics XL device comprising data acquisition software and software for exploiting the results. The fluorometry measurements are made for each sample.

The same procedure is followed with the erythrocytes from blood group B.

This operating mode is used with three different batches of IgG (B3) and is also applied to three different batches of IgG prepared by ethanol fractionation according to the Cohn method (cited above) (B1).

The results obtained appear in the following Table 3

TABLE 3

| Samples | Anti-A antibody content | Anti-B antibody content |
|---|---|---|
| B1 | 1 | 1 |
| B2 (3 batches) | 3.80; 3.55; 3.59 | 3.80; 4.45; 3.31; |
| B3 (3 batches) | 0.66; 0.68; 0.69 | 0.33; 0.73; 0.50 |

### Example 4

### Measurement of the Haemolysis of the Erythrocytes

An approximately 1% (v/v) suspension of papainated erythrocytes of blood group A, B, AB or O is prepared and is then counted in a Malassez cell in order to obtain approximately 10 erythrocytes. approximately 100 μCi of 51Cr (1 volume per 1 volume of erythrocytes) is added. The whole is incubated for approximately 1 hour and the radiomarked erythrocytes are then washed 5 times.

The radiomarked erythrocytes are then put in contact with samples of IgG concentrates (B2) obtained at example 1, at a concentration of approximately 1.2 mg/ml for approximately 5.106 radiomarked erythrocytes, in a volume of approximately 100 μl.

A volume approximately identical to the previous one of approximately 100 μl of normal serum of blood group AB is then added to the previous mixture in order to add the various factors of the remainder.

The reaction mixture obtained is then incubated for approximately 4 hours at a temperature of approximately 37° C.

The reaction mixture is then centrifuged for approximately 1 minute at approximately 2000 revolutions/min, and a measurement is made of the radioactivity of the incubated supernatant solution, using suitable commercially available devices. The measured radioactivity of the solution is proportional to the degree of haemolysis of the erythrocytes treated and, consequently, to the anti-A and anti-B antibody content. An identical procedure is followed with erythrocytes in blood groups B, AB and O, all being rhesus+, and with a sample of serum of group O+. This operating mode is used with three different batches of IgG (B2). In addition, the method is applied to three batches of commercial samples of IgG concentrates, denoted C2 to C4, and a sample C5 of serum of group O+included as a negative reference.

The measured radioactivity of the solution is proportional to the degree of haemolysis of the erythrocytes treated and consequently to the quantity of anti-A and anti-B antibodies fixed to the erythrocytes.

The haemolysis results obtained are presented in Table 4 below

TABLE 4

| Degree of | | | | | |
|---|---|---|---|---|---|
| haemolysis of | | | | | |
| erythrocytes (%) | B3 | C2 | C3 | C4 | C5 |
| Group A+ | 6 | 16 | 13 | 30 | 34 |
| | 6.2 | 15.5 | 13.5 | 31 | 34.4 |
| | 6.5 | 16.3 | 13.2 | 31 | 34.6 |
| Group B+ | 5 | 13.1 | 11.2 | 25 | 34 |
| | 4.8 | 13.3 | 10.8 | 25.3 | 34.4 |
| | 5.4 | 13.4 | 10.9 | 25.4 | 34.6 |
| Group AB+ | 6 | 16 | 15.1 | 31 | 34 |
| | 5.9 | 15.7 | 15.1 | 31.4 | 34.4 |
| | 6.5 | 16.3 | 15.4 | 30.9 | 34.6 |
| Group O+ | 0.1 | 0.2 | 0.22 | 0.33 | 2 |
| | 0.15 | 0.25 | 0.24 | 0.32 | 2.2 |
| | 0.12 | 0.21 | 0.24 | 0.30 | 2.7 |

The results obtained show that the IgG concentrate B3 that was subjected to affinity chromatography according to the invention contains the smallest quantity of AcaA and AcaB given that the degree of haemolysis of the erythrocytes coming from the different blood groups is the lowest. No haemolysis is observed with erythrocytes of phenotype O+, included as a negative reference.

### Example 5

### Measurement of the Polyreactivity of the IgG Concentrates B2 (Before Immunoaffinity Chromatography) and after this Chromatography (IgG Concentrate B3) Described in Example 1

The polyreactivity of these IgG concentrates is measured in accordance with patent EP 1 059 088, which is incorporated by reference, using two antigens that react with the polyreactive IgGs. These are myosin and albumin modified by dinitrophenyl groups (DNP albumin).

Table 5 presents the polyreactive IgG enrichment factors of samples B2, B3 and C4 of example 3, the IgG content of which was fixed arbitrarily at 1, by way of reference.

These measurements were made on three different batches of IgG concentrates considered.

TABLE 5

| Sample | Myosin | DNP albumin |
|---|---|---|
| B1 | 1 | 1 |
| B2 (3 batches) | 1.2; 0.8; 1.2 | 3.2; 1.0; 2.0 |
| B3 (3 batches) | 0.4; 0.4; 0.4 | 1.0; 1.0; 1.0 |
| C4 (3 batches) | 2.0; 2.0; 2.0 | 8.0; 6.0; 7.0 |

The results indicate that the IgG concentrate B3 of the invention contains approximately 5 to 8 times less polyreactive IgGs than the concentrate of the prior art C4.

### Example 6

### Comparative Example on the Efficacy of the IgG Concentrates (B3) Depleted in AcaA, AcaB and Polyreactive IgGs, with IgG Concentrates (B1)

The study related to mice deficient in Fc RI and Fc RIII receptors treated with a view to evaluating the immunomodulating activity of the IgG concentrates according to an embodiment. These animals serve as a model for a thrombocytopenic purpura. An IgG concentrate (B1) obtained by ethanol fractionation according to Cohn is used as a reference.

The experimental protocol described by Teeling J. L. et al (Blood, 15/08/2001, vol. 98, number 4, pp. 1095-1099 [10]), which is incorporated by reference, is applied. The platelets, collapsed by injection of antiplatelet monoclonal IgGs from approximately 9.10⁸/ml to the level of approximately 2.10⁸/ml, rise again to approximately 7.10⁸/ml in animals treated with the IgG B1 and B3 concentrates at a therapeutic dose of approximately 1 g/kg.

The immunomodulating activity of the IgG concentrate B3 according to an embodiment was not modified by use of immunoaffinity chromatography.

### Example 7

### Example of Implementation

Evaluation of the dose to be administered and of the tolerance to treatment.

The IgG composition for implementing an embodiment, for example the B3 composition or another IgG composition available on the market, is administered to neonates, boys and girls, aged less than approximately 28 days, suffering from ABO haemolytic disease, the symptoms of which are hyperbilirubinemia, a positive direct neonatal antiglobulin test (direct Coombs test) and a high reticulocyte count (greater than or equal to approximately 10%).

The patient exclusion criteria are a deficiency of IgA, the presence of anti-IgE/IgG antibodies, the indication of an exchange transfusion on birth with a level of bilirubin in the umbilical cord greater than or equal to approximately 4 mg, a foetoplacental anasarca (hydrops fetalis), cardiac insufficiency, and prenatal treatment (maternal IVIG and/or in utero transfusion).

The treatment protocol is an administration of IgG at approximately 500 mg/kg and 2000 mg/kg (n=approximately 10), alone or in combination with phototherapy, able to be reiterated.

The primary evaluation criteria are the need for an exchange transfusion, and the total level of bilirubin in the serum is measured approximately 3 days after the first administration of the composition.

The main secondary evaluation criteria are the total duration of the phototherapy, the duration of the hospitalisation time, the development of a late anaemia at the 27^{th} day post partum, and the total bilirubin level in the serum approximately 24 hours after the first administration of the immunoglobulin composition.

The comparative analysis of the results is carried out by means of the Kaplan Meier method.

The stop rule is the initiation of an exchange transfusion in accordance with the directives of the clinical practices of the American Academy of Paediatric Subcommittee on Hyperbilirubinemia (Paediatrics. 2004 July; 114(1): 297-316), which is incorporated by reference.

The bilirubin level is measured, by one of the known tests, so as to monitor the change in remission of the patient.

From the foregoing it will be appreciated that, although specific embodiments have been described herein for purposes of illustration, various modifications may be made without deviating from the spirit and scope of the disclosure. Furthermore, where an alternative is disclosed for a particular embodiment, this alternative may also apply to other embodiments even if not specifically stated.

### LIST OF REFERENCES

[1] Cohn et al, 1946, J. Am. Chem. Soc. 68, 459; Oncley et al. 1949, J. Am. Chem. Soc. 71, 541.
[2] Gottstein et al, (2003) Archives of Disease in Childhood: Fetal and Neonatal Edition, vol. 88, no. 1, p. F6-F10.
[3] Alplay et al, (1999), Acta Paediatrica, International Journal of Paediatrics; vol. 88, no. 2, p. 216-219.
[4] Nasseri et al, (2006) Saudi Med J.; 27(12): 1827-30.
[5] Steinbuch et al. (1969) Ref. Franc. Et. Clin, and Biol, XIV, 1054.
[6] Buchta C. et al, Biologicals, 33, 2005, 41-48.
[7] Wilson J. R. et al, Muscle & Nerve, 29(9), 1997, 1142-1145.
[8] Copelan E. A. et al, Transfusion, 26, 1986.410-412.
[9] Misbah S. A. et al, Drug Safety, 9, 1993, 254-262.
[10] Teeling J. L. et al, Blood 15/08/2001, vol. 98, number 4, pp. 1095-1099.

All of the above references are incorporated by reference.

1. Immunoglobulin G (IgG) composition for therapeutic use, comprising respective anti-A and anti-B antibody titers in accordance with a negative result to the indirect Coombs test (method 2.6.20 of the European Pharmacopoeia), as a medicament for the treatment of neonatal jaundice caused by maternal-foetal incompatibility with respect to the ABO system or of neonatal haemolytic disease caused by ABO incompatibility.

2. Composition according to claim 1, said respective anti-A and anti-B antibody titers in accordance with a negative result to the indirect Coombs test being 64, reverse dilution (“results given as a whole number (the lower part of the dilution fraction)”).

3. Composition according to claim 1, said respective anti-A and anti-B antibody titers in accordance with a negative result to the indirect Coombs test being between 0 and 8, reverse dilution (“results given as a whole number (the lower part of the dilution fraction)”).

4. Composition according to claim 1, said indirect Coombs test being carried out with an IgG solution with an initial concentration adjusted to 30 g/l.

5. Composition according claim 1, having a residual polyreactive IgG content of between 0.01% and 0.1% with respect to the total IgG content.

6. Composition according to claim 1, further comprising stabilizers.

7. Composition according to claim 1, in which the stabilizers represent a mixture of an alcohol sugar, preferably mannitol or sorbitol, glycine and non-ionic detergent.

8. Composition according to claim 1, injectable intravenously.

9. Composition according to claim 1, obtainable by a method comprising the following steps:
a) preparation of an IgG concentrate by ethanol fractionation and/or by chromatographic separation, comprising a viral inactivation step,
b) immunoaffinity chromatography by percolation of said IgG concentrate on a mixture of carriers, the matrices of which are grafted with oligosaccharide groups antigenically similar to blood groups A and B, and
c) elimination filtration of viruses and/or particles with a size greater than 20 nm.

10. Composition according to claim 9, step a) of said method comprising a prepurification by precipitation of lipid contaminants using a blood plasma or a fraction of blood plasma enriched with IgG, a single chromatography on an anion exchange resin carrier carried out at alkaline pH and a selective elution of the IgGs in one step using a suitable buffer with a pH of between 4 and 7.

11. Composition according to claim 9, the oligosaccharide groups of said method representing trisaccharides corresponding to the epitopes of blood groups A and B.

12. Composition according to claim 8, the viral inactivation step of said method being performed by solvent/detergent.

13. Composition according to claim 8, said mixture of grafted carriers of groups antigenically similar to blood group A and to blood group B being in respective proportion of between 25/75 and 75/52 (v/v), preferably 50/50 (v/v) in each of said carriers.

14. Composition according to claim 8, said method comprising steps of concentration by ultrafiltration and sterilising filtration.

15. Composition according to claim 14, said sterilising filtration step being performed by nanofiltration.

16. Composition according to claim 9 said method comprising, after step c), a step of adding preservation stabilizers to said IgG concentrate.

17. Use of a composition according to claim 1, for manufacturing a medicament intended for the treatment of neonatal jaundice caused by maternal-foetal incompatibility with respect to the ABO system.

18. Method of treating neonatal jaundice caused by maternal-foetal incompatibility with respect to the ABO system or ABO neonatal haemolytic disease, comprising the administration, to an person in need thereof, of an IgG composition as defined in claim 1.
